(19) United States
(12) Patent Application Publication
(10) Pub. No.: US 2024/0393313 A1
(43) Pub. Date: Nov. 28, 2024

(54) Method for Extracting Black-Odorous Water Body Based on Cart Classification Model

(71) Applicants: NORTH CHINA INSTITUTE OF AEROSPACE ENGINEERING, Langfang, Hebei (CN); AEROSPACE INFORMATION RESEARCH INSTITUTE, CHINESE ACADEMY OF SCIENCES, Haidian District, Beijing (CN); GUANGZHOU UNIVERSITY, Guangzhou, Guangdong (CN)

(72) Inventors: Qichao ZHAO, Langfang, Hebei (CN); Xingfa GU, Haidian District, Beijing (CN); Guohong LI, Langfang, Hebei (CN); Jiaguo LI, Haidian District, Beijing (CN); Wenhao ZHANG, Langfang, Hebei (CN); Jinnian WANG, Guangzhou, Guangdong (CN); Yongtao JIN, Langfang, Hebei (CN); Wenlong HAN, Langfang, Hebei (CN)

(21) Appl. No.: 18/272,918

(22) PCT Filed: Jan. 9, 2023
(86) PCT No.: PCT/CN2023/071317

§ 371 Date: Jul. 18, 2023

(30) Foreign Application Priority Data
Jan. 11, 2022 (CN) 202210027897.1

(51) Int. Cl. G01N 33/18; G01N 21/31; G01N 21/77

(52) U.S. Cl. CPC G01N 33/1826; G01N 21/31; G01N 21/77; G01N 2021/7773; G01N 2201/129

(57) ABSTRACT

A method for extracting a black-odorous water body based on a CART classification model includes: selecting a research region, designing sampling points within the region; monitoring relevant chemical indicators of the water body at various sampling points, extracting remote sensing reflectance data of the water body, determining a type of the water body according to a classification standard of relevant chemical indicators for an urban black-odorous water body; comparing and analyzing the remote sensing reflectance data to obtain spectral change features of the black-odorous water body and a general water body; constructing each node of a decision tree according to the spectral change features and based on Gini index, constructing a decision tree classification model to obtain classification results of the black-odorous water body and the general water body, calculating a classification accuracy; analyzing the classification results to obtain spatiotemporal distribution changes of black-odorous water bodies in the region.

### CROSS-REFERENCE TO RELATED APPLICATION

This application claims the priority of Chinese Patent Application No. 202210027897.1 filed with the China National Intellectual Property Administration on Jan. 11, 2022, and entitled “METHOD FOR EXTRACTING BLACK-ODOROUS WATER BODY BASED ON CART CLASSIFICATION MODEL”, the disclosure of which is incorporated by reference herein in its entirety.

### TECHNICAL FIELD

The present disclosure relates to the technical field of environment monitoring, and in particular to a method for extracting a black-odorous water body based on a Classification & Regression Tree (CART) classification model.

### BACKGROUND

Black-odorous water body is an extreme phenomenon caused by severe organic pollution of natural water bodies. The early research on the black-odorous water body mainly focused on chemical mechanism, such as the influence of specific pollutants on black and odor of rivers, the causes of black-odorous phenomenon of water bodies, black-odorous indexes, and black-odorous water body evaluation. However, in the field of remote sensing, the use of remote sensing means for analyzing, monitoring and evaluating the black-odorous water body is still in its infancy.

At present, the research foundation of remote sensing identification of black-odorous water bodies in China is still weak, few successful cases have been reported, and only Beijing, Nanjing, Shenyang and Taiyuan have conducted more systematic research and application. As most of the above work is based on the spectral difference between the black-odorous water body and clean water body in visible light band, through the construction of typical spectral feature indexes, the black-odorous water body is identified using a threshold segmentation method. For example, Wen Shuang et al. observed measured samples in Nanjing, compared various combinations of bands, and pointed out that a band ratio method can be used to better identify black-odorous water body: Yao Yue et al. constructed a BOI index (black-odor index) based on the band ratio method to identify black-odorous water bodies in Shenyang; Shen et al. thought that a purity method can be used to better identify different types of black-odorous water bodies; Li Jiaqi et al. carried out experiments and verifications in Taiyuan, proposed that a WCI index (water cleanliness index) can be used to better distinguish black-odorous water bodies from general water bodies, and also considered that when extracting black-odorous water bodies, the remote sensing interpretation marks (e.g., water color, water body width, the presence or absence of garbage dump, etc.) of water bodies also need to be combined besides spectral features, thus achieving better results. These methods have achieved good results when used in some large-scale regions, but there are still some shortcomings in the research and application of remote sensing identification of black-odorous water bodies, which are mainly reflected in the following aspects.

(1) Threshold selection relies excessively on expert experience and knowledge. In most of the above methods, empirical thresholds are set based on the observation of small samples, which is easily interfered by operators and samples. thus leading to non-objective and inconsistent threshold settings, which are prone to controversy.

(2) A single feature is used for classification in most of the above methods, but in many cases, since the spectral features of some black-odorous water bodies are similar to those of general water bodies, it is difficult to distinguish the black-odorous water bodies from the general water bodies directly using the single feature, resulting in low accuracy in identifying black-odorous water bodies from general water bodies.

### SUMMARY

To this end, an objective of some embodiments of the present disclosure is to provide a method for extracting a black-odorous water body based on a CART (classification and regression tree) classification model, which is used for solving the problems of insufficient objectivity in threshold setting of the black-odorous water body and less feature selection at present.

In order to achieve the above objective, the present disclosure provides the following solution.

A method for extracting a black-odorous water body based on a Classification and Regression Tree (CART) classification model, including the following steps:
step 1): selecting a research region, and designing multiple sampling points within the research region:
step 2): monitoring relevant chemical indicators of the water body at various sampling points. respectively, extracting remote sensing reflectance data of the water body, sending the relevant chemical indicators and the data to a laboratory, and determining a type of the water body according to a classification standard of relevant chemical indicators for an urban black-odorous water body, where the relevant chemical indicators include transparency, dissolved oxygen, oxidation-reduction potential, and ammonia nitrogen:
step 3): comparing and analyzing the remote sensing reflectance data extracted at the various sampling points to obtain spectral change features of the black-odorous water body and a general water body, where the spectral change features include a reflectance difference value between a green band and a red band, a reflectance value of a near infrared band, and a sum of reflectance values of a visible light band:
step 4): constructing each node of a decision tree according to the spectral change features and based on a Gini index, constructing a decision tree classification model to obtain classification results of the black-odorous water body and the general water body, and calculating a classification accuracy; and
step 5): analyzing the classification results to obtain spatiotemporal distribution changes of black-odorous water bodies in the research region.

Alternatively, the multiple sampling points in step 1) are designed according to a principle of random distribution.

Alternatively, the classification standard of urban black-odorous water body in step 2) at least satisfies one of relevant chemical indicator requirements as follows: transparency in a range of 0 cm to 25 cm, dissolved oxygen in a range of 0 mg/L to 2 mg/L, oxidation-reduction potential in a range of 0 mV to 50 mV, and ammonia nitrogen of not less than 8 mg/L.

Alternatively, a calculation formula of the Gini index in step 4) is:

$$Gini ( p ) = {\sum }_{k = 1}^{K} {p}_{k} ( 1 - {p}_{k} ) = 1 - {\sum }_{k = 1}^{K} {p}_{k}^{2} ;$$

where K is a number of contained categories, and px is a probability of a k-th category.

According to specific embodiments provided by the present disclosure, the following technical effects are obtained: in accordance with the method for extracting a black-odorous water body based on a CART classification model provided by the present disclosure, scientific research is conducted on training threshold and multi-feature black-odorous water body remote sensing identification, and an objective and standard technical system is established, thus reducing the uncertainty brought by subjective experience. Further, high-accuracy remote sensing identification of black-odorous water bodies is achieved by adopting multi-feature classification.

### BRIEF DESCRIPTION OF THE DRAWINGS

To describe the technical solutions in the embodiments of the present disclosure or in the prior art more clearly, the following briefly introduces the accompanying drawings required for describing the embodiments. Apparently, the accompanying drawings in the following description show merely some embodiments of the present disclosure, and those of ordinary skill in the art may still derive other drawings from these accompanying drawings without creative efforts.

FIG. 1 is a flow chart of a method for extracting a black-odorous water body based on a CART classification model according to an embodiment of the present disclosure;

FIG. 2 is a distribution diagram of sampling points according to an embodiment of the present disclosure;

FIG. 3A is a diagram illustrating remote sensing reflectance of a general water body according to an embodiment of the present disclosure;

FIG. 3B is a diagram illustrating remote sensing reflectance of a black-odorous water body according to an embodiment of the present disclosure;

FIG. 4A is a diagram illustrating Rrs(R)−Rrs(G) feature curve according to an embodiment of the present disclosure;

FIG. 4B is a diagram illustrating Rrs(B)+Rrs(G)+Rrs(R) feature curve according to an embodiment of the present disclosure;

FIG. 4C is a diagram illustrating Rrs(NIR) feature curve according to an embodiment of the present disclosure;

FIG. 5 is a diagram illustrating classification by a CART model according to an embodiment of the present disclosure;

FIG. 6 is a diagram illustrating a classification result from a CART model according to an embodiment of the present disclosure;

FIG. 7 is a grid diagram illustrating distribution of black-odorous water bodies in August 2018 according to an embodiment of the present disclosure;

FIG. 8 is a grid diagram illustrating distribution of black-odorous water bodies in April 2021 according to the embodiment of the present disclosure.

### DETAILED DESCRIPTION OF THE EMBODIMENTS

The technical solutions in the embodiments of the present disclosure will be described below clearly and completely with reference to the accompanying drawings in the embodiments of the present disclosure. Apparently, the described embodiments are merely a part rather than all of the embodiments of the present disclosure. All other embodiments obtained by those of ordinary skill in the art based on the embodiments of the present disclosure without creative efforts shall fall within the protection scope of the present disclosure.

To this end, an objective of some embodiments of the present disclosure is to provide a method for extracting a black-odorous water body based on a CART classification model, which is used for solving the problems of a lack of enough objectivity in threshold setting of the black-odorous water body and less feature selection at present.

To make the above-mentioned objective, features and advantages of the present disclosure more apparently, the present disclosure will be further described in detail below in conjunction with the accompanying drawings and the specific embodiments.

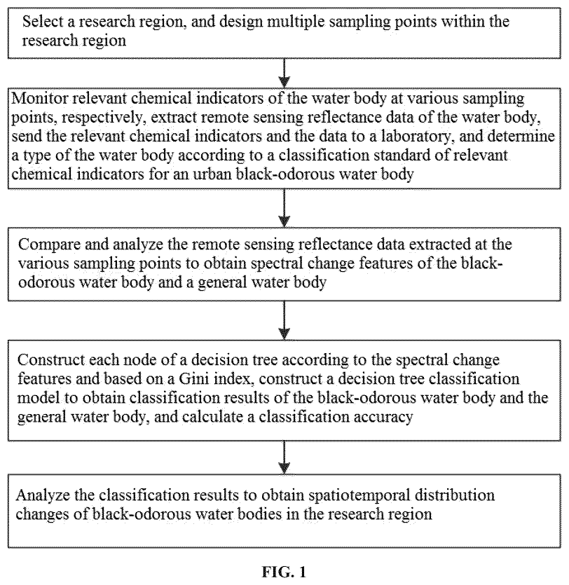

As show in FIG. 1, a method for extracting a black-odorous water body based on a CART classification model according to an embodiment of the present disclosure includes the following steps 1)-5).

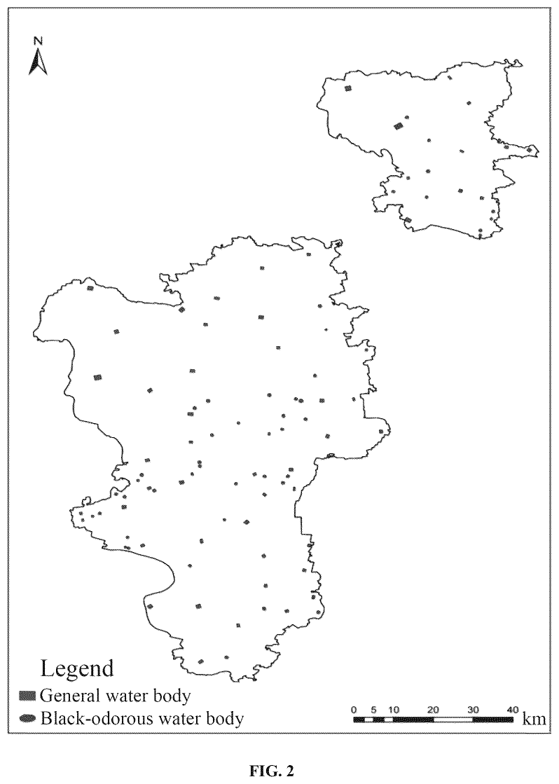

In step 1), a research region is selected, and multiple sampling points are designed within the research region. As shown in FIG. 2, by taking Langfang City, Hebei Province, China, as an example, the sampling points are designed with the principle of random distribution.

In step 2), relevant chemical indicators for a water body are monitored at each sampling point, respectively, remote sensing reflectance data of the water body are extracted and then sent to a laboratory together with the relevant chemical indicators, and a type of water body is determined according to a classification standard of relevant chemical indicators for an urban black-odorous water body. The relevant chemical indicators include transparency (SD), dissolved oxygen (DO), oxidation-reduction potential (ORP), and ammonia nitrogen (NH3-N). The classification of the urban black-odorous water bodies is as shown in Table 1.

TABLE 1
Classification of urban black-odorous water bodies

| | Black-odorous |
|---|---|
| Feature indicator | water body |
| Transparency cm | 0 to 25 |
| Dissolved oxygen mg/L | 0 to 2 |
| Oxidation-reduction potential mV | 0 to 50 |
| Ammonia nitrogen mg/L | Not less than 8 |

In a case that a water body at a sampling point at least satisfies one of the relevant chemical indicator requirements as follows: transparency in a range of 0 cm to 25 cm, dissolved oxygen in a range of 0 mg/L to 2 mg/L, oxidation-reduction potential in a range of 0 mV to 50mV, and ammonia nitrogen of not less than 8 mg/L, the water body at the sampling point is a black-odorous water body.

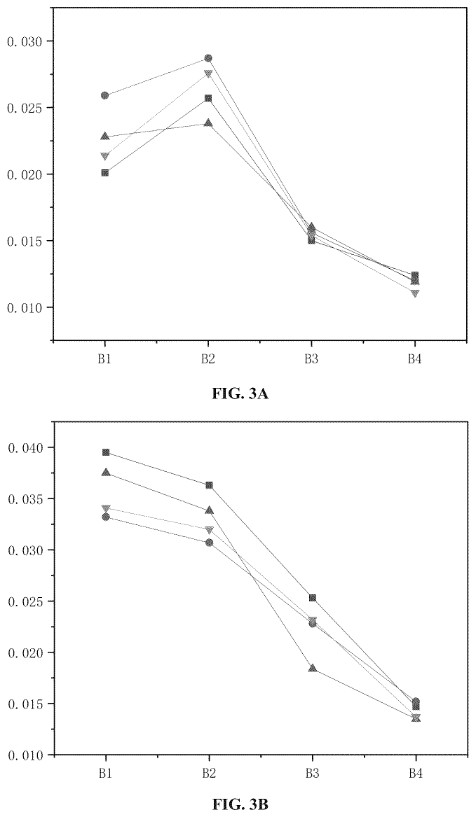

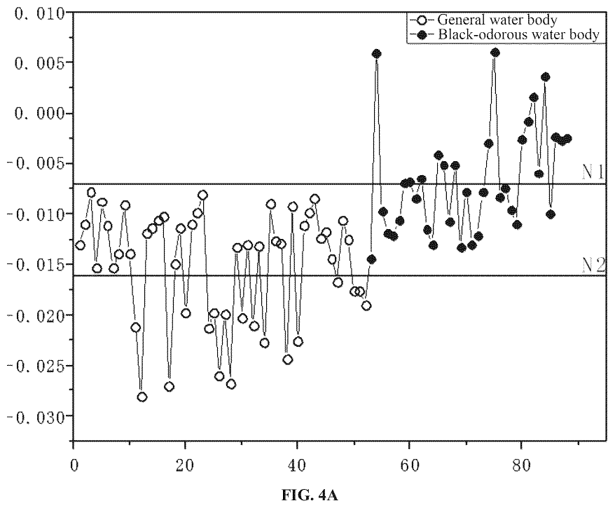

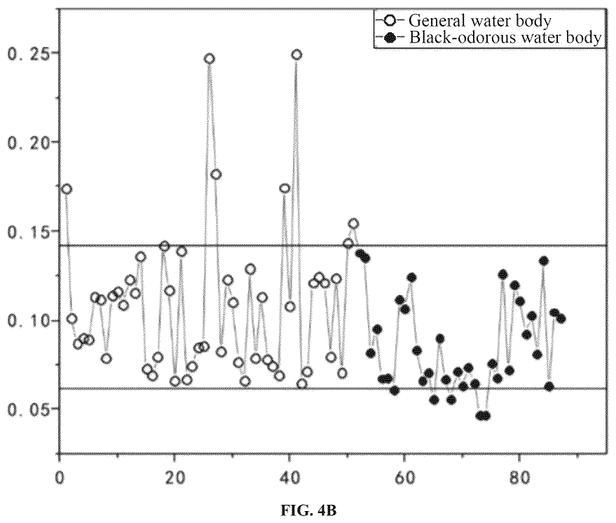

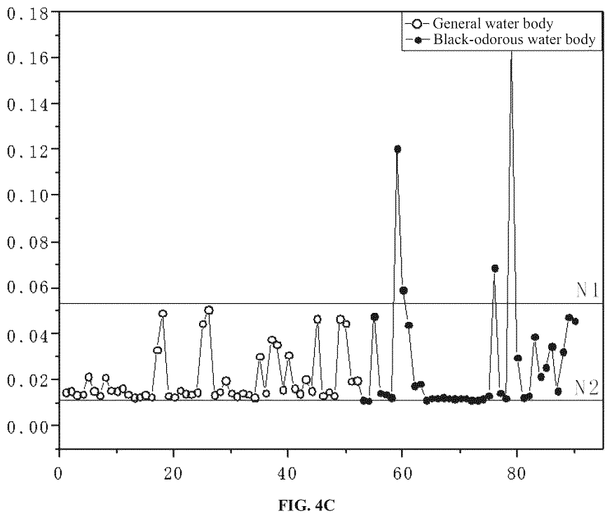

In step 3), the remote sensing reflectance data extracted at various sampling points are compared and analyzed to obtain spectral change features of the black-odorous water body and a general water body, where the spectral change features include a reflectance difference value between a green band and a red band, a reflectance value of a near infrared band, and a sum of reflectance values of a visible light band. Corresponding remote sensing reflectance results are obtained according to the sampling point information, as shown in FIG. 3A and FIG. 3B. By comparison, it can be seen that the overall reflectance of the black-odorous water body is relatively low, and the spectral curve change from the green band to the red band is relatively smooth. In addition, the reflectance in the near infrared band of the general water body is relatively low, and the reflectance in the near infrared band is lower than that of the visible light band. However, some samples of the black-odorous water bodies have higher reflectance in the near infrared band, which is different from the general water body. The reflectance of these samples in the near infrared band is obviously higher than that in the visible light band. As shown in FIG. 3A and FIG. 3B, the vertical ordinate is remote sensing reflectance, B1 denotes a blue band, B2 denotes a green band, B3 denotes a red band, and B4 denotes a near infrared band. Therefore, Rrs(R)−Rrs(G) as a reflectance difference value between the green band and the red band, is adopted to reflect a curve gentleness in such a band range, where Rrs(R) is the reflectance in the red band, Rrs(G) is the reflectance in the green band, as shown in FIG. 4A. Rrs(B)+Rrs(G)+Rrs(R) is a sum of reflectances in the visible light band, reflecting the feature that the overall reflectance of the black-odorous water body is low, as shown in FIG. 4B, where Rrs(B) is the reflectance in the blue band. Some black-odorous water bodies show high reflectance in the near infrared band, so the reflectance Rrs(NIR) in the near infrared band is added to the feature combination, as shown in FIG. 4C. The vertical ordinate in FIG. 4A is a difference value sr⁻¹ between the reflectances of the red band and the green band, the vertical ordinate in FIG. 4B is the sum sr⁻¹ of remote sensing reflectance in the red band. remote sensing reflectance in the blue band and remote sensing reflectance in the green band, and the vertical ordinate in FIG. 4C is the remote sensing reflectance sr⁻¹ in the near infrared band.

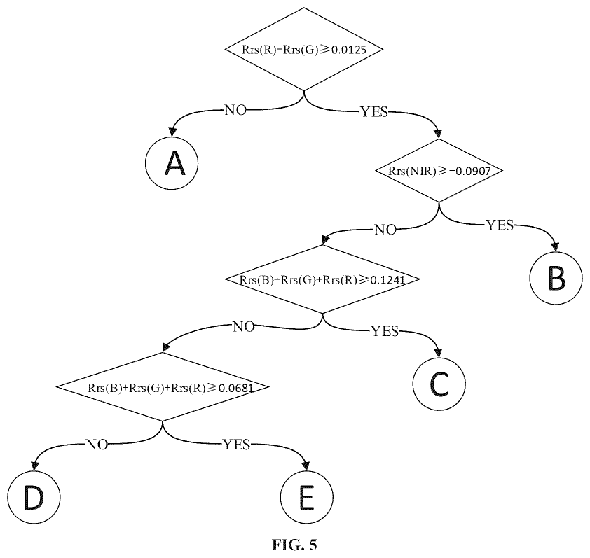

In step 4), each node of a decision tree is constructed according to the spectral change features and based on the Gini index, as shown in FIG. 5, and in turn a decision tree (CART) classification model is constructed to obtain classification results of the black-odorous water body and a general water body, and the classification accuracy is calculated according to the corresponding results in Table 2, so as to obtain a corresponding confusion matrix and a kappa coefficient:

TABLE 2
Classification and membership categories

| Classification | | | |
|---|---|---|---|
| result | Membership | Zone | Category |
| A | 1 | Confidence zone | Black-odorous water |
| B | 0.78 | Fuzzy zone | Black-odorous water y |
| C | 0.75 | Fuzzy zone | Black-odorous water y |
| D | 0.2 | Fuzzy zone | General water body |
| E | 0 | Confidence zone | General water body |

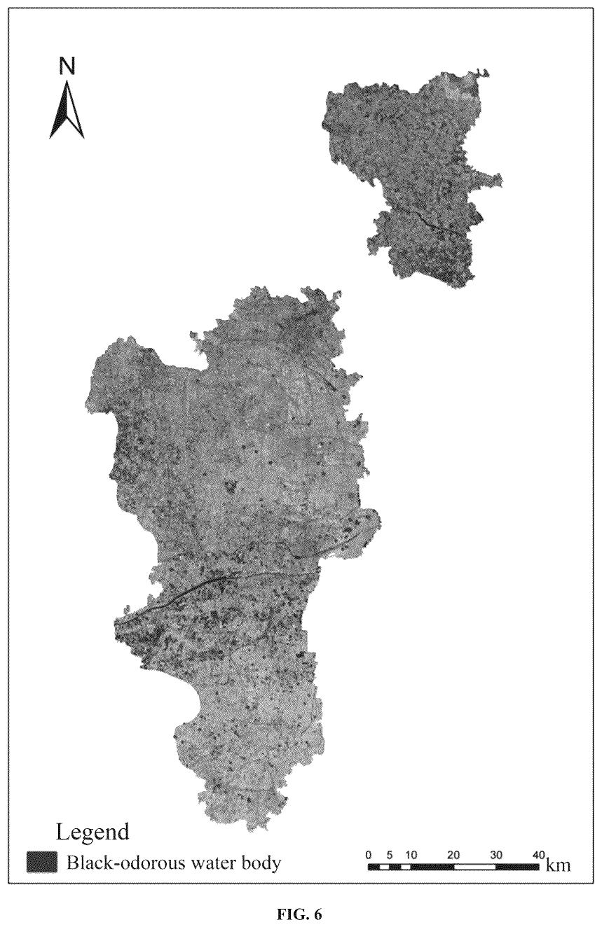

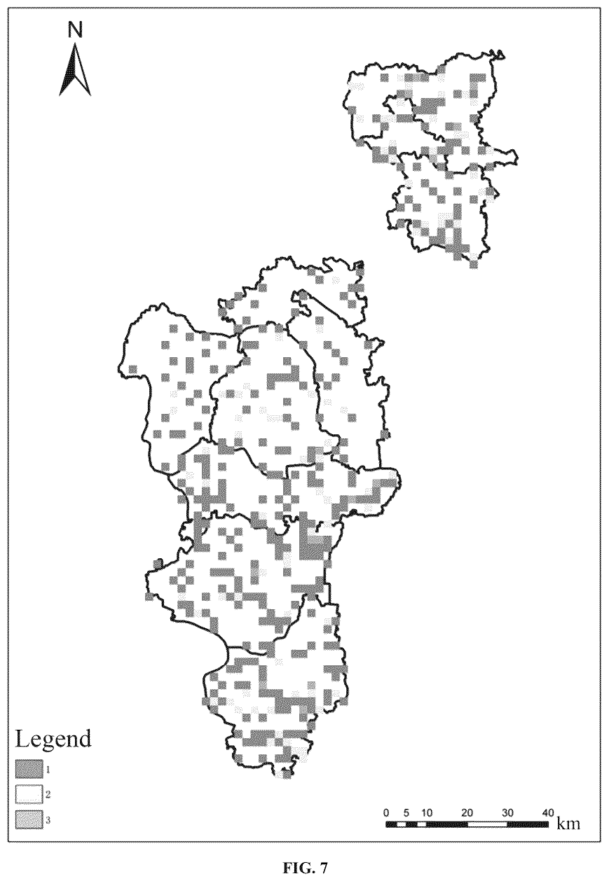

According to the classification results in Table 2, twenty-six sampling points were extracted from a confidence zone, twenty-four of which were determined correctly, thereby obtaining an accuracy of 92.30%; twenty sampling points were extracted from a fuzzy zone, sixteen of which were determined correctly, thereby obtaining an accuracy of 80.00%; therefore, the overall accuracy is 86.95%. Further, the obtained confusion matrix is shown in Table 3, and the kappa coefficient is 0.7281, indicating that the data have good consistency and the model accuracy is good. As shown in FIG. 6, according to the classification results, the distribution of black-odorous water bodies in Langfang City, Hebei Province, China is obtained in conjunction with remote sensing images.

TABLE 3
Comparison of extraction between confidence zone and fuzzy zone

| | General | Black-odorous | General | Black-odorous | |
|---|---|---|---|---|---|
| | water | water | water | water | |
| | body in | body in | body in | body in | |
| | confidence | confidence | fuzzy | fuzzy | |
| | zone | zone | zone | zone | Sum |
| Correct | 16 | 7 | 0 | 8 | 31 |
| judgment | | | | | |
| False | 1 | 2 | 9 | 3 | 15 |
| judgment | | | | | |
| Sum | 17 | 9 | 9 | 11 | 46 |

A calculation formula of the Gini index in step 4) is:

$$Gini ( p ) = {\sum }_{k = 1}^{K} {p}_{k} ( 1 - {p}_{k} ) = 1 - {\sum }_{k = 1}^{K} {p}_{k}^{2} ,$$

where Gini(p) is the calculated Gini index, for a sample set D, the included category is k, and the probability that the sample belongs to the k-th category is denoted as pₖ. The extraction of black-odorous water body discussed in the embodiments of the present disclosure is a typical binary classification problem, namely, K=2:

Gini (p)=2p(1−p), where p=p₁ or p=p₂, p₁ indicates the probability that the category is the general water body, p₂ indicates the probability that the category is the black-odorous water body.

In step 5), the classification results are analyzed to obtain spatiotemporal distribution changes of black-odorous water bodies in the research region. In accordance with the present disclosure, for the same research region, the distribution of the black-odorous water bodies at different times is obtained through the CART classification model, and variations of the distribution of the black-odorous water bodies in the same research region over time is obtained by comparing the distribution of the black-odorous water bodies at different times.

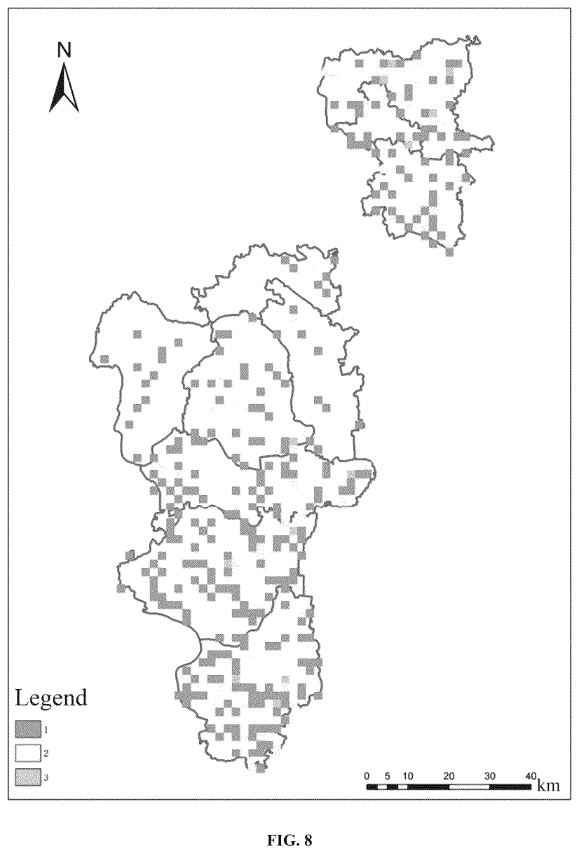

According to the remote sensing monitoring results in April 2021, the spatial distribution features of black-odorous water bodies in Langfang City, Hebei Province, China were analyzed, and the classification results were resampled to 20 km×20 km. Afterwards, the number of black-odorous water bodies in each grid was counted, as shown in FIG. 7 and FIG. 8, to obtain hotspot grid maps of the black-odorous water bodies in Langfang City, Hebei Province, China in August 2018 and April 2021. According to the distribution of hotspot grids, the high-density distribution areas of black-odorous water bodies in Langfang City are mainly concentrated in the north, mid-eastern and south of Langfang city, with the reasons as follows: on one hand, the above three areas are relatively low-lying, with large pit and pond bases; on the other hand, the livestock breeding and food processing industries in Sanhe city and Dachang County in the north of Langfang City are relatively developed, the mid-east and south of Langfang City have a high concentration of township enterprises and relatively high population density, so the production and life activities involved in sewage discharge in the above three areas are more intense, which also increases the probability of black-odorous water bodies.

In accordance with the method for extracting a black-odorous water body based on a CART classification model provided by the present disclosure, scientific research is conducted on training threshold and multi-feature black-odorous water body remote sensing identification, and an objective and standard technical system is established, thus reducing the uncertainty brought by subjective experience. Further, high-accuracy remote sensing identification of black-odorous water bodies is achieved by adopting multi-feature classification.

Embodiments of the present specification are described in a progressive manner, each embodiment focuses on the difference from other embodiments, and the same and similar parts between the embodiments may refer to each other.

In this specification, some specific embodiments are used for illustration of the principles and implementations of the present disclosure. The description of the foregoing embodiments is used to help illustrate the method of the present disclosure and the core ideas thereof. In addition, those of ordinary skill in the art can make various modifications in terms of specific implementations and the scope of application in accordance with the ideas of the present disclosure. In conclusion, the content of this specification shall not be construed as limitations to the present disclosure.

1. A method for extracting a black-odorous water body based on a Classification and Regression Tree (CART) classification model, comprising the following steps:
step 1): selecting a research region, and designing a plurality of sampling points within the research region;
step 2): monitoring relevant chemical indicators of the water body at various sampling points, respectively, extracting remote sensing reflectance data of the water body, sending the relevant chemical indicators and the data to a laboratory, and determining a type of the water body according to a classification standard of relevant chemical indicators for an urban black-odorous water body, wherein the relevant chemical indicators comprise transparency, dissolved oxygen, oxidation-reduction potential, and ammonia nitrogen;
step 3): comparing and analyzing the remote sensing reflectance data extracted at the various sampling points to obtain spectral change features of the black-odorous water body and a general water body, wherein the spectral change features comprise a reflectance difference value between a green band and a red band, a reflectance value of a near infrared band, and a sum of reflectance values of a visible light band;
step 4): constructing each node of a decision tree according to the spectral change features and based on a Gini index, constructing a decision tree classification model to obtain classification results of the black-odorous water body and the general water body, and calculating a classification accuracy; and
step 5): analyzing the classification results to obtain spatiotemporal distribution changes of black-odorous water bodies in the research region.

2. The method according to claim 1, wherein the plurality of sampling points in step 1) are designed according to a principle of random distribution.

3. The method according to claim 1, wherein the classification standard of urban black-odorous water body in step 2) at least satisfies one of relevant chemical indicator requirements as follows: transparency in a range of 0 cm to 25 cm, dissolved oxygen in a range of 0 mg/L to 2 mg/L, oxidation-reduction potential in a range of 0 mV to 50 mV, and ammonia nitrogen of not less than 8 mg/L.

4. The method according to claim 1, wherein a calculation formula of the Gini index in step 4) is:
$$Gini ( p ) = {\sum }_{k = 1}^{K} {p}_{k} ( 1 - {p}_{k} ) = 1 - {\sum }_{k = 1}^{K} {p}_{k}^{2} ;$$
wherein K is a number of contained categories, and pₖ is a probability of a k-th category.
